(19) United States
(12) Patent Application Publication
(10) Pub. No.: US 2009/0318377 A1
(43) Pub. Date: Dec. 24, 2009

(54) MEDICAGENIC ACID SAPONIN AND USES THEREOF

(75) Inventors: Louis-Philippe Vezina, Neuville (CA); Nathalie Landry, St. Jean-Chrysostome (CA); Joelle Pelletier, Augustin-de-Desmaures (CA); Sylvain Savard, L'Ancienne-Lorette (CA)

Correspondence Address: SCHWEGMAN, LUNDBERG & WOESSNER, P.A., P.O. BOX 2938, MINNEAPOLIS, MN, 55402, US

(73) Assignee: MEDICAGO INC., SAINTE-FOY, QUEBEC (CA)

(21) Appl. No.: 12/373,476

(22) PCT Filed: Jul. 13, 2007
(86) PCT No.: PCT/CA2007/001255

§ 371 Date: Sep. 1, 2009

Related U.S. Application Data
(60) Provisional application No. 60/830,655, filed on Jul. 13, 2006.
(60) Provisional application No. 60/886,768, filed on Jan. 26, 2007.

(51) Int. Cl. A61K 31/704; A61P 3/06

(52) U.S. Cl. 514/33

(57) ABSTRACT

A cholesterol-lowering preparation comprising medicagenic acid saponin is disclosed. The amount of medicagenic acid saponin in the preparation is greater than 50% by weight to produce the cholesterol-lowering effect in an animal. A method of purifying a preparation of at least 30% medicagenic acid saponin is also disclosed. The preparation may be purified from alfalfa plants and used as a treatment for lowering cholesterol and triglycerides in an animal, in particular a human.

### FIELD OF INVENTION

The present invention relates to medicagenic acid saponin and uses thereof, and in particular medicagenic acid saponin purified from Medicago sativa (alfalfa).

### BACKGROUND OF THE INVENTION

High levels of plasma cholesterol in humans increase the risk of Coronary Heart Diseases, which is the major cause of morbidity and mortality throughout the United States and most industrialized countries. Several drugs are available to manage hypercholesterolemia and hyperlipidemia such as HMG-CoA reductase inhibitors (statins) and ezetimibe, a newly approved selective cholesterol absorption inhibitor. The long-term use of statins does not always allow an optimal blood cholesterol level to be reached and may induce undesirable side effects. There is therefore a need to develop new compounds with complementary mechanism of action.

Sapogenins are sterol derivatives widely distributed in plants. They occur naturally as their O-glycoside derivatives, saponins. Saponins are therefore ampiphilic compounds consisting of a non-polar moiety, sapogenins, and glycosidic polar side chains. It has been shown that their ampiphilic nature allows saponins to bind to various lipid constituents (including plasma lipids such as cholesterol) and increase their solubility.

Saponins occur in several plant species, with beans, soybean and alfalfa being the plant species showing the highest content (w/w). Several hundred different types of saponins have been isolated from different plant species. They differ mainly in the structure of the aglycone moiety, the nature and the number of carbohydrate moieties linked to the agycone and by their different hydroxyl and carboxyl groups.

Plant preparations enriched in their saponin content have been tested as dietary supplement for their ability to modify the content of plasma lipids in mammals, including humans. In most of the early studies, the relative abundance of saponins in the fed preparations was generally lower than 20% (w/w), with other active plant constituents such as flavonoids being present in high amounts, making it difficult to draw clear conclusions as to the role of saponins in the control of blood lipids.

Kim et al. [1] disclose that saponins partly purified from the plant species Yucca schidigera and Quillaja saponaria reduced blood cholesterol by an average of 13% in hypercholesterolemic patients.

Steroid saponins isolated from fenugreek seeds were tested in a rat model. Petit et al. [4] reported a 20% reduction of plasma cholesterol in animals fed with 12.5 mg of saponins per 300 g body weight for 4 weeks. Matsuura [3] assessed saponins isolated from garlic as modifiers of the risk of cardiovascular diseases. Garlic saponins were fractionated and preparations containing from 0.1-17% (w/w) in saponins were administered to hypercholesterolemic rats at doses ranging from 0.003-3 g per kg of body weight per day. Authors reported up to a 60% reduction in plasma cholesterol after 16 weeks for some saponin fractions.

Saponins were also reported for their anti-obesity effect. In one experiment, crude saponins from Korean Red Ginseng showed an anti-obesity effect in diet-induced obesity in rats (Kim et al. [6]). Rats received daily intraperitoneal injections of crude saponins at 200 mg/kg. Authors reported a 27% body weight reduction in the treated group compared to control.

Medicago sativa (alfalfa) is a plant species showing a high content in saponins, up to 2-3% dry weight in leaves and up to 30% dry weight in roots (Massiot et al. [7] and Hanson et al. [8]). Alfalfa also contains a uniquely high relative abundance of a group of acidic saponins, medicagenic acid glycosides. FIG. 1 shows the different sapogenins found in alfalfa species. Soyasapogenol, medicagenic acid and zhanic acid glycosides are the most abundant saponins found in alfalfa foliage, representing around 40%, 17% and 19% of total saponin content (Nowacka et al. [9]).

Since alfalfa has a high content in saponins, numerous animal and human studies have also been conducted with alfalfa preparations to demonstrate their potential in the control of cholesterolemia and general lipidemia. There again, the preparations were only modestly enriched (an average of 2% (w/w)) and contained complex mixtures of saponins and other molecular components with known biological activities.

Houston et al. [10] reported the different studies conducted with alfalfa preparations. They are summarized in Table 1.

TABLE 1
Summary of animal and clinical studies conducted using alfalfa saponins (TC: Total Cholesterol;
HDL: High-Density Lipoprotein; LDL: Low- Density Lipoprotein; TG: Triglycerides).

| | | STUDY DURATION | | |
|---|---|---|---|---|
| | | AND SAPONIN | EVALUATED | |
| | | PREPARATION | DAILY DOSE | |
| STUDY | SUBJECTS | USED | OF SAPONINS | RESULTS |
| Beaumont | Human | 40 g of alfalfa seeds 4 | 530 mg | Type II patients showed a |
| et al [11] | hyperliproteimia | times per day for 8 | | 25% reduction in plasma |
| | patients of type II | weeks | | cholesterol. |
| | and IV | | | |
| Rashaf et | Rats | Preparation showing | n/d | Reduction in cholesterol |
| al [12] | | 2% (w/w) in saponins | | production from the liver |
| | | | | and increased excretion of |
| | | | | cholesterol in feces. |
| Molgaard | Humans with | 40 g of alfalfa seeds 3 | 400 mg | TC −26%, LDL −30% |
| et al [13] | moderate | times per day for 8 | | |
| | hypercholesterole | weeks | | |
| | mia | | | |
| Ilona C. | Humans with | 1 g of Esterin processed | 52 mg during 2 | TC −28%, HDL +11%, |
| [14] | moderate | alfalfa 3 times per day | weeks followed | LDL −28% |
| | hypercholesterole | for 2 weeks followed by | by 35 mg for 4 | |
| | mia | 1 g 2 times per day for 4 | months | |
| | | months | | |
| Ilona C. | Humans with | 1 g of Esterin processed | 35 mg* | TC −20%, HDL +13%, |
| [15] | moderate | alfalfa 2 times per day | | TC −16%, no toxicity |
| | hypercholesterole | for 3 months | | |
| | mia | | | |
| Golub | Human | 1 g of Esterin processed | 35 mg | TC −24%, HDL +35%, |
| [16] | | alfalfa 2 times per day | | LDL −29%, TG −15% |
| | | for 16 weeks | | |
| Czeizel | Humans with | 2 g of Esterin processed | 35 mg | TC −11.7-22%, HDL +25-50%, |
| [17] | moderate | alfalfa per day for 12 | | LDL −20-37%, |
| | hypercholesterole | weeks | | TG −4%, no toxicity |
| | mia | | | |
| Szigetti | Humans with | 2 g of Esterin processed | 70 mg | Women: TC −25%, |
| [18] | moderate | alfalfa 2 times per day | | HDL +25%, TG −20% |
| | hypercholesterole | for 3 months | | Men: TC −21%, |
| | mia | | | HDL +33%, TG −25% |
| | | | | 90% of patients had a |
| | | | | body weight reduction |
| | | | | ranging from 4.4 to 8.8 |
| | | | | pounds |

These studies clearly show a cholesterol-lowering effect of alfalfa-derived preparations containing saponins. As the preparations contained a mixture of saponins and other natural chemicals, a large dose of the preparations was required to achieve the desired effect in each of these studies. For example, in the study by Ilona C. [15], a 1 g dose of esterin-processed alfalfa was required twice daily to provide an estimated 35 mg daily dose of mixed saponins. The esterin processed alfalfa of the study by Ilona C. [15], sold under the trade name Cholestaid™ has an undesirable off-taste and may induce certain side effects such as diarrhea and periods of eructation. Furthermore, Cholestaid™ may not be formulated into any food formulation. More recently, purified saponin preparations were tested demonstrating their role in the management of hypercholesterolemia and hyperlipidemia effect.

Lee et al. [2] studied the metabolism of soyasaponin B isolated from soyabeans and fed to Syrian Golden hamster at an average daily dose of 128 mg/kg for 4 weeks. The authors reported a reduction in plasma cholesterol by 20%, a 33% reduction in high-density lipoprotein (HDL) and an 18% reduction in triglycerides. The study also showed that greater production of soyasaponin B metabolites was associated with better plasma cholesterol status, suggesting that gut microbial variation in soyasaponin metabolism may influence the health benefit of these saponins.

Platycodin saponins isolated from the roots of Platycodon grandiflorum were also evaluated as an obesity and hyperlipidemia treatment (Zhao et al. [3]). The saponin preparation used in this study consisted of 7 different saponins, bearing sugar moieties on one two or three carbons of the aglycone moiety. The daily oral administration of 30 or 70 mg/kg of this platycodin saponin preparation resulted in a body weight reduction of 13%, which was correlated to the food intake restriction. Plasma triglycerides were lowered by 24-28%, and low-density lipoprotein (LDL) were decreased by 41-52%. No significant changes in plasma cholesterol and HDL were noticed.

Saponins of different nature also have different chemico-physical properties in mixed solutions. Acidic saponins (including medicagenic acid saponins), but not neutral saponins, will bind cholesterol in vitro, and neutral saponins complex with bile salts more easily than do acidic saponins.

### SUMMARY OF THE INVENTION

The present invention relates to medicagenic acid saponin and uses thereof, and in particular medicagenic acid saponin purified from Medicago sativa (alfalfa).

It is an object of the invention to provide an improved preparation having cholesterol-lowering effect.

According to the present invention there is provided a preparation comprising from about 50% to about 100% by weight medicagenic acid saponin. Preferably the preparation comprises at least 80% by weight medicagenic acid saponin. The preparation comprising medicagenic acid saponin is preferably purified from Medicago sativa (alfalfa).

The present invention pertains to a preparation as just defined, wherein at least 50% of the medicagenic acid saponin is selected from the group consisting of 3-GlcA 28-Xyl-Rha-Ara-MA (medicagenic acid), 3-GlcA 28-Rha-Ara-MA, and a combination thereof.

The present invention further provides a method of producing a preparation comprising at least 50% medicagenic acid saponin from plant material comprising:
(i) providing a solution of said plant material;
(ii) filtering said solution to obtain a filtrate;
(iii) performing solid phase extraction (SPE) on said filtrate to obtain a SPE elute;
(iv) performing chromatography on said SPE elute to produce said preparation.

The present invention pertains to a method as just defined, wherein the plant material in step (i) is from Medicago sativa (alfalfa). Chromatography in step (iv) is preferably performed using a high performance liquid chromatography (HPLC) system. The medicagenic acid saponin preparation may be eluted from the HPLC system at about 20 to about 40 minutes, or any time therebetween. Preferably the medicagenic acid saponin preparation is eluted from the HPLC system at about 25 to about 35 minutes.

The present invention provides a preparation produced by the method of the present invention comprising from about 50% to about 100% by weight, or preferably at least 80% by weight, medicagenic acid saponin. At least 30%, or preferably 50% of the medicagenic acid saponin in the preparation produce by the method of the present invention may comprise 3-GlcA 28-Xyl-Rha-Ara-MA (medicagenic acid), 3-GlcA 28-Rha-Ara-MA, or a combination thereof. The preparation produced by the method of the present invention preferably does not contain any phyto-estrogen coumestrol.

The present invention further provides a method of lowering cholesterol and triglycerides comprising administering a preparation comprising from about 50% to about 100% by weight medicagenic acid saponin to an animal, preferably a human. The preparation preferably comprises at least 80% by weight medicagenic acid saponin. The preparation comprising medicagenic acid saponin is preferably purified from Medicago sativa (alfalfa). At least 30%, or preferably 50% of the medicagenic acid saponin in the preparation may be selected from the group consisting of 3-GlcA 28-Xyl-Rha-Ara-MA (medicagenic acid), 3-GlcA 28-Rha-Ara-MA, and a combination thereof. The preparation may also be used to manage hypercholesterolemia and hyperlipidemia in an animal

There is further provided by the present invention, use of a preparation comprising at least 50% by weight medicagenic acid saponin, for lowering cholesterol in an animal preferably a human. The preparation preferably comprises at least 80% by weight medicagenic acid saponin. The preparation comprising medicagenic acid saponin is preferably purified from Medicago sativa (alfalfa). At least 30% of the medicagenic acid saponin in the preparation may be selected from the group consisting of 3-GlcA 28-Xyl-Rha-Ara-MA (medicagenic acid), 3-GlcA 28-Rha-Ara-MA, and a combination thereof.

The present invention pertains to a use of a preparation comprising from about 50% to about 100% by weight medicagenic acid saponin, for lowering cholesterol in an animal. Preferably the animal is a human.

The present invention therefore provides a method of purifying medicagenic acid saponin from plant material, in particular alfalfa plant material. The purified medicagenic acid saponin preparation can be used to lower cholesterol, triglycerides, or both, in an animal. A significant cholesterol lowering effect can be attributed to the medicagenic acid saponin. By using a purified preparation of medicagenic acid saponin, a reduced amount of the preparation is required to bring about a cholesterol-lowering effect than other saponin preparations. The use of a lower amount of saponin when compared to that used in the prior art, to achieve a similar biological effect, allows the medicagenic saponin preparation of the present invention to be formulated as a pharmaceutical. The purified preparation of medicagenic acid saponins generally has a better taste than other saponin preparations and can also be used as a food supplement or for neutraceutical or functional foods.

The present invention further pertains to a preparation comprising a medicagenic acid saponin (preferably from about 50% to about 100% by weight medicagenic acid saponin), for use in medicine. More specifically the present invention also provides use of a preparation comprising a medicagenic acid saponin (preferably from about 50% to about 100% by weight medicagenic acid saponin) in the manufacture of a medicament for treatment of hypercholesterolemia and/or hyperlipidemia.

There is further provided by the present invention a foodstuff comprising a food material and a medicagenic acid saponin. The medicagenic acid saponin may be in a purified form and in particular the medicagenic acid saponin may be purified from Medicago sativa. In one aspect 30% to about 100% of the medicagenic acid saponin is selected from the group consisting of 3-GlcA 28-Xyl-Rha-Ara-MA (Medicagenic Acid), 3-GlcA 28-Rha-Ara-MA, and a combination thereof. For example the medicagenic acid saponin may be selected from the group consisting of 3-GlcA 28-Xyl-Rha-Ara-MA (Medicagenic Acid), 3-GlcA 28-Rha-Ara-MA, and a combination thereof.

It will be appreciated that the method of producing the foodstuff is encompassed here. Thus, there is further provided by the present invention a method of producing a foodstuff comprising providing a food material; providing a preparation comprising from about 50% to about 100% by weight medicagenic acid saponin; combining the food material and the preparation to provide the foodstuff.

A similar cholesterol-lowering effect was achieved in hamsters treated with the purified medicagenic acid saponin preparation of the present invention as was achieved in hamster treated with soyasaponin B isolated from Soya beans (Lee et al. [2]), however the dose of the medicagenic acid saponin preparation required to achieve this effect was much lower than the dose required to achieve a similar effect using soyasaponin B. A lower dose of medicagenic acid saponin preparation is therefore advantageously required to bring about a significant cholesterol-lowering effect.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows common sapogenins found in Medicago sativa (alfalfa) including medicagenic acid saponin;

FIG. 2A shows a Total Ion Count from analytical mass spectrometry chromatogram of the medicagenic acid saponins product produced in accordance with an embodiment of the present invention; FIG. 2B shows the fragmentation pattern of medicagenic acid saponin analyzed using mass spectrometry

FIG. 3 shows the effect of medicagenic acid saponin treatment on food intake. The Control Group of hamsters (▴) were fed with a normal rodent diet; the Placebo Group of hamsters (⋄) were fed with a High Fat High Cholesterol (HFHC) diet and received a placebo of salin; and the Saponin Group of hamsters (▪) were fed with a High Fat High Cholesterol (HFHC) diet and received the purified medicagenic acid saponin produced in accordance with the present invention. Six animals were used for each group except the Saponin Group.

FIG. 4 shows the effect of medicagenic acid saponin treatment (25 mg/kg/day) on plasma lipid concentrations after 4 weeks. The average triglycerides, total cholesterol, high-density lipoprotein (HDL) and non-HDL for the Control Group of hamsters fed with a normal rodent diet (Control); the Placebo Group of hamsters fed with a High Fat High Cholesterol (HFHC) diet and a placebo of saline (HFHC+placebo); and the Saponin Group of hamsters fed with a High Fat High Cholesterol (HFHC) diet and 25 mg/kg/day purified medicagenic acid saponins produced in accordance with the present invention (HFHC+saponin), are shown.

FIG. 5 shows the effect of medicagenic acid saponin treatment (75 mg/kg/day) on plasma lipid concentrations after 3 weeks. The average triglycerides, total cholesterol, high-density lipoprotein (HDL) and non-HDL for the Control Group of hamsters fed with a normal rodent diet (Control); the Placebo Group of hamsters fed with a Moderate Cholesterol (MC) diet and a placebo of saline (MC+placebo); and the Saponin Group of hamsters fed with a Moderate Cholesterol (MC) diet and 75 mg/kg/day purified medicagenic acid saponins produced in accordance with the present invention (MC+saponin), are shown.

FIG. 6 shows the plasma cholesterol levels measured weekly for each group of hamsters shown in FIG. 4, which were fed a normal rodent diet for a wash-out period of 3 weeks after the 4 week treatment period.

### DETAILED DESCRIPTION

The present invention relates to medicagenic acid saponin and uses thereof, and in particular medicagenic acid saponin purified from Medicago sativa (alfalfa).

The following description is of a preferred embodiment.

The present invention provides a cholesterol-lowering preparation comprising medicagenic acid saponin. The preparation may also be used to manage hypercholesterolemia and hyperlipidemia in an animal. The preparation of medicagenic acid saponin may be used as a food supplement, or for neutraceutical or functional foods.

The amount of medicagenic acid saponin in the cholesterol-lowering preparation is from about 70% to about 100% by weight, or any amount therebetween. This amount produces a cholesterol-lowering effect in an animal. The preparation may contain between about 75% to about 99.5% by weight, or any amount therebetween of medicagenic acid saponin, for example, 72%, 74%, 76%, 78%, 80%, 82%, 84%, 86%, 88%, 90%, 92%, 94%, 96% and 98% by weight medicagenic acid saponin, or any amount therebetween. In a preferred embodiment, the preparation comprises from about 80% to about 100% by weight, or any amount therebetween, of medicagenic acid saponin.

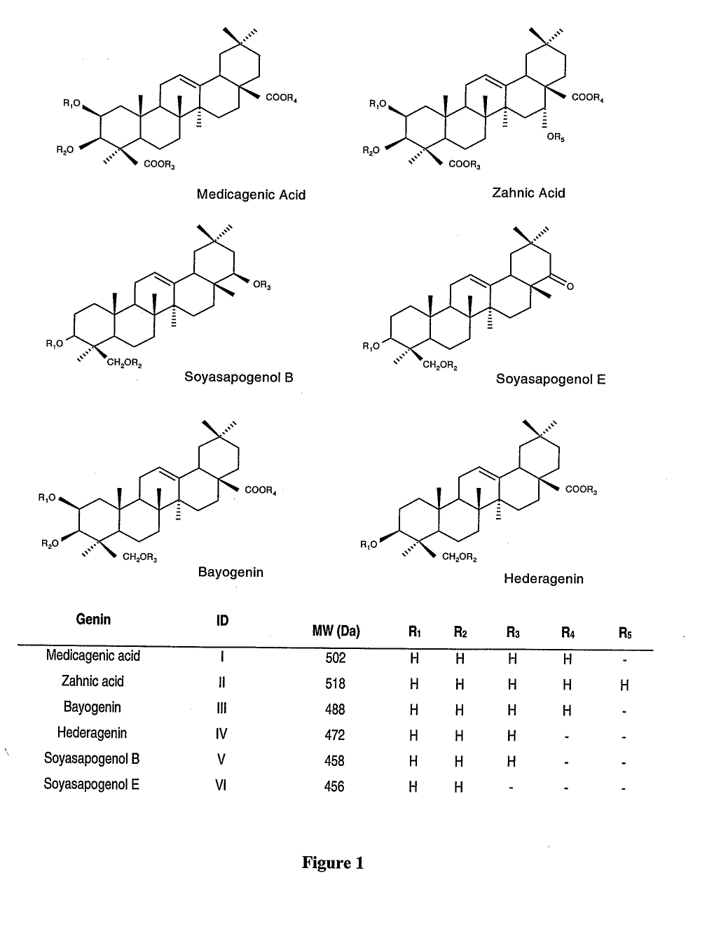

The medicagenic acid saponin is preferably purified from Medicago sativa (alfalfa). FIG. 1 shows the different sapogenins found in alfalfa species. Soyasapogenol, medicagenic acid and zhanic acid glycosides are the most abundant saponins found in alfalfa foliage, representing around 40%, 17% and 19% of total saponin content respectively (Nowacka et al. [9]).

By the term “medicagenic acid saponin” it is meant a saponin having the formula of medicagenic acid as shown in FIG. 1.

Medicagenic acid saponins have an additional acidic group when compared with soyasaponin B (Lee et al. [2]), and platycodin saponins, (Zhao et al. [3]). Without wishing to be bound by theory, the additional acidic group may confer different therapeutic properties when compared to other pure saponins tested in vivo. Table 2 (see Examples) shows the structure of different medicagenic acid saponins that may be found in the preparation of the present invention. Preferably, at least 50% of the medicagenic acid saponin in the preparation of the present invention is selected from the group consisting of 3-GlcA 28-Xyl-Rha-Ara-MA (Saponin 1 in Table 2), 3-GlcA 28-Rha-Ara-MA (Saponin 2 in Table 2), or a combination thereof. For example, from about 50% to about 100% or any amount therebetween of the medicagenic acid saponin in the preparation of the present invention may be selected from the group consisting of 3-GlcA 28-Xyl-Rha-Ara-MA (Saponin 1), 3-GlcA 28-Rha-Ara-MA (Saponin 2), or a combination thereof.

By the term “lowering cholesterol” it is meant reducing one or more markers of cholesterol selected from the group consisting of total cholesterol, plasma triglycerides, non-HDL, and a combination thereof. The amount of marker may be determined using standard techniques as would be known to one of skill in the art.

The present invention further provides a method of producing a preparation comprising at least 70% by weight, or any amount therebetween, medicagenic acid saponin, for example from about 70% to about 100% by weight, or any amount therebetween, from plant material comprising:
(i) providing a solution of said plant material;
(ii) filtering said solution to obtain a filtrate;
(iii) performing solid phase extraction (SPE) on said filtrate to obtain a SPE elute comprising a mixture of saponins;
(iv) performing chromatography on said SPE elute to produce said preparation.

The plant material is preferably from an alfalfa plant and may comprise alfalfa flour. The alfalfa plant material may be suspended in an aqueous solution, such as, but not limited to a solvent, for example ethanol, methanol and the like, and filtered to remove any residue. Any liquid in the filtrate may then be evaporated at atmospheric pressure or under vacuum. More than one filtration may be performed on the solution of plant material, a non-limiting example being that the plant material solution may first be press filtered, followed by a second filtration using a finer filter (for example, but not limited to, a 20 μm filter) to obtain a filtrate typically comprising saponin concentrated extract.

The filtrate is then loaded onto a solid phase extraction (SPE) column for example a silica C18-10% carbon column, or other C-18 columns of different carbon percentage and different matrix chemistry, as would be known to one of skill in the art. The column may be preconditioned with a solvent, such as, but not limited to ethanol or methanol, and may be further preconditioned with ethanolic solution or the like. Elution of saponins may be achieved using an aqueous ethanolic solution or the like. The elute may then be evaporated to dryness under vacuum.

Separation of medicagenic acid saponins from other saponins and plant components may be achieved by chromatography or other similar system. The preferred chromatography system used in the method of the present invention is a high performance liquid chromatography (HPLC) system. For example, which is not to be considered limiting in any manner, the HPLC system may include the use of a Spherisorb (Waters Corporation, Milford, Mass.) column, equilibrated with a mixture comprising water, acetonitrile, acetic acid, and eluted with a gradient of increasing acetonitrile. However, other elution mixtures may be also as would be known to one of skill in the art. The medicagenic acid saponins of interest may be eluted, for example, under room temperature conditions involving: 0-2 min isocratic (80% water, 20% acetonitrile, both containing 0.01% acetic acid), followed by a 2-30 min linear gradient, from 20 to 39% acetonitrile, a 30 to 50 min linear gradient from 39 to 45% acetonitrile, and a 50 to 56 min linear gradient from 45 to 98% acetonitrile, delivered at a flow rate of 14 mL/min. Under these conditions, medicagenic acid saponins of interest will elute at about 20 to about 40 minutes, or any time therebetween, for example about 21, 22, 23, 24, 25, 26, 27, 28, 29, 30, 31, 32, 33, 34, 35, 36, 37, 38 and 39 minutes or any time therebetween. Preferably the preparation of medicagenic acid saponin is eluted at about 25 to about 35 minutes. As would be readily understood by one of skill in the art, the use of alternate solvents will effect the time and elution profile of the medicagenic saponins.

The preparation produced by the method of the present invention may contain between about 50% to about 100% by weight medicagenic acid saponin or any amount therebetween, for example, at least 50%, 55%, 60%, 65%, 70%, 72%, 74%, 76%, 78%, 80%, 82%, 84%, 86%, 88%, 90%, 92%, 94%, 96% and 98% by weight medicagenic acid saponin, or any amount therebetween. At least 30%, for example at least 30%, 35%, 40%, 45%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98% or more, or any percentage therebetween of the medicagenic acid saponin in the preparation may comprise 3-GlcA 28-Xyl-Rha-Ara-MA (Saponin 1 in Table 2), 3-GlcA 28-Rha-Ara-MA (Saponin 2 in Table 2), or a combination thereof.

As herein described in the examples, a sample of 3.5 kg of alfalfa flour may produce approximately 7 g of solid preparation containing greater than 80% medicagenic acid saponins following the method of the present invention. The preparation produced by the method of the present invention preferably does not contain any phyto-estrogen coumestrol, for example containing less than about 1%, or for example, from about 1% to about 0.01% or any amount therebetween by weight of phyto-estrogen coumestrol. Medicagenic acid saponin preparations comprising less than 0.5% of phyto-estrogen coumestrol, are considered phyto-estrogen coumestrol free.

The purified medicagenic acid saponin preparation may be used to lower cholesterol in an animal, for example, but not limited to a human.

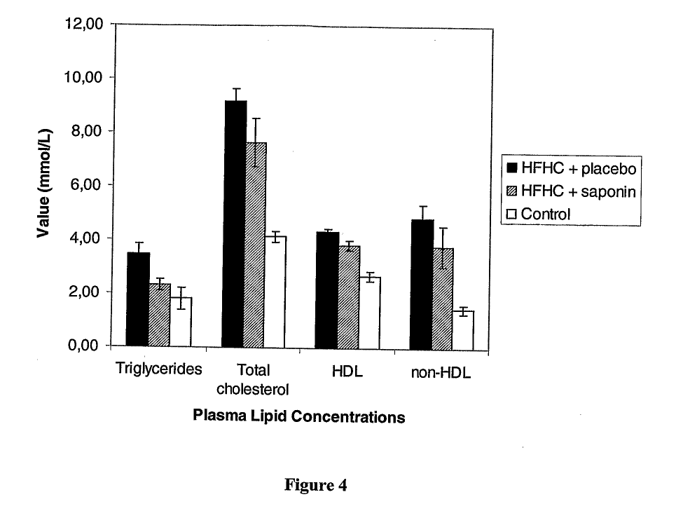

As herein described in the examples, hamsters fed with a High Fat High Cholesterol (HFHC) diet containing 0.5% cholesterol and 20% of energy as fat, to induce hypercholesterolemia and then given purified medicagenic acid saponin preparation prepared using the method of the present invention, as a non-limiting example, a dose of 25 mg/kg by gavage once a day for a period of 4 weeks, showed significantly reduced total cholesterol, plasma triglycerides and high-density lipoprotein (HDL) (by 17%, 33% and 11.4% respectively) compared to hamsters fed the HFHC diet and given a saline placebo (FIG. 4). These results are comparable to the cholesterol lowering effect of soyasaponin B isolated from soyabeans fed to Syrian Golden hamster at an average daily dose of 128 mg/kg for 4 weeks (Lee et al. [2]), which showed a reduction in plasma cholesterol by 20%, a 33% reduction in high-density lipoprotein (HDL) and an 18% reduction in triglycerides. However, the purified medicagenic acid saponin preparation of the present invention, is able to produce a comparable cholesterol-lowering effect in animal studies at a much lower dose than soyasaponin B (i.e. 25 mg/kg medicagenic acid saponin preparation per day compared to 128 mg/kg soyasaponin B per day) up to about 20% of the dose required of soyasaponin B. Therefore, dosages of medicagenic acid saponin from about 10% to about 100% or any amount therebetween of that of soyasaponin B, may be used to achieve a similar biological response as that obtained using soyasaponin B.

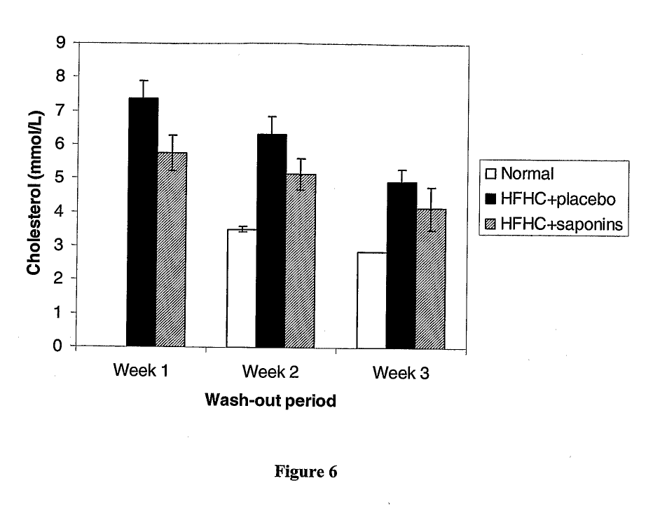

Medicagenic acid saponins of the present invention may have an effect on the elimination of body-synthesized cholesterol. As shown in the examples described herein, when hamsters were given a regular rodent diet containing no cholesterol, following diet induced hypercholesterolemia and treatment with either medicagenic acid saponins or a placebo, the hamsters previously treated with medicagenic acid saponins were shown to have a significantly lower plasma cholesterol levels than the hamsters previously given a placebo, for a period of two weeks following the treatment period (FIG. 6). Therefore, the preparation of the present invention comprising medicagenic acid saponins may be used to enhance the elimination of plasma cholesterol.

Furthermore, as herein described in the examples, hamsters fed with a Moderate Cholesterol (MC) diet containing 0.25% cholesterol and 12% of energy fat that received medicagenic acid saponins purified in accordance with the present invention at a dose of approximately 75 mg/kg administered orally once a day for 3 weeks, showed a significant reduction in total cholesterol and non-HDL (by 16.5%, and 17.5% respectively) and a 39% reduction in plasma triglycerides compared to hamsters fed with a MC diet and given a placebo for the same treatment period. This indicates that even in moderate hypercholesterolemia, the medicagenic acid saponins of the present invention show a cholesterol lowering effect.

The present invention therefore further provides a method of lowering cholesterol comprising administering the preparation of the present invention comprising medicagenic acid saponins, to an animal, preferably a human.

The present invention, also provides a use of the preparation of the present invention comprising medicagenic acid saponins, for lowering cholesterol in an animal preferably a human.

As discussed herein, there is provided by the present invention a foodstuff comprising a food material and a medicagenic acid saponin. There is also provided by the present invention a method of producing a foodstuff comprising providing a food material; providing a preparation comprising from about 50% to about 100% by weight, or any amount therebetween, for example 50, 55, 60, 65, 70, 75, 80, 85, 90, 95 100% by weight or any amount therebetween, medicagenic acid saponin; combining the food material and the preparation to provide the foodstuff. The term “foodstuff” as used in the present specification and claims refers generally to edible products and beverages of the food and feed industry. The edible products in question are products in which the medicagenic acid saponin may be incorporated such that the beneficial effects described herein are provided to the consumer of the foodstuff in addition to the nutritive food material.

Although taste provided the medicagenic acid saponin should be minimal so as not to be readily detectable, in some aspects is it desirable and provides advantageous properties. In one aspect the medicagenic acid saponin is provided from alfalfa in a form such that flavour is imparted. In one aspect the flavour is a “grassy note”. In one aspect the medicagenic acid saponin is combined with a flavouring of or extract from maple, cocoa, raisin, prune, and/or caramel.

Typical foodstuffs are raw and cooked meat, ready to eat meals, pasta sauces, pasteurised soups, mayonnaise, salad dressings, marinades, oil-in-water emulsions, margarines, low fat spreads, water-in-oil emulsions, dairy products, cheese spreads, processed cheese, dairy desserts, flavoured milks, cream, fermented milk products, cheese, butter, condensed milk products, ice cream mixes, soya products, pasteurised liquid egg, bakery products, confectionery products, fruit products, and foods with fat-based or water-containing fillings, salad dressings, acidic dairy products (including natural cheese, cottage cheese, acidified cheese, cream cheese, yoghurt, sour cream, processed cheese), fruit juices, acidic drinks, alcoholic drinks (including wine and beer), chilled dough and cooked or uncooked bakery products, dairy fillings and toppings for baked goods, surface glazes and coatings for bakery items and other heat-processed items, condiments, dips, purees, pickles, marinades, marinated meat or poultry, breaded meat or poultry, pizza toppings and bases, fast food products, kits for making snacks or meals, kits for making bakery products, pet food, broiler feed and any other acidic, heat-processed and/or fungal fermented food products.

For example the foodstuff may be a juice beverage, such a fruit juice or fruit juice based beverage.

The foodstuff may contain medicagenic acid saponin in a purified form. This has been found to be particularly advantageous since impurities which may effect the properties of the foodstuff, such as the stability of the foodstuff or the taste of the foodstuff.

In one aspect of the invention the medicagenic acid saponin present in the foodstuff is purified from Medicago sativa. The medicagenic acid saponin may be purified using any suitable technique as would be known to one of skill in the art including for example preparing an aqueous extract from Medicago sativa, obtaining a filtrate from the extract, performing solid phase extraction (SPE) on the filtrate and obtaining an a SPE elute, and performing chromatography on the SPE elute to obtain the medicagenic acid saponin. Alternatively, the medicagenic acid saponin may be purified using supercritical fluid extraction (SFE; or supercritical solvent extraction) involving placing plant material into a pressure vessel and pumping a liquefied gas or solvent through the material in the vessel at specific pressure and temperature. A non-limiting example of a liquefied gas or solvent includes CO², ethyl alcohol, ethanol, propane, isobutene, dimethyl ether, R134a or other refrigerant gases. Non-limiting examples of low temperature (low pressure) cold extraction involves a loading rates of about 1-40 volumes liquefied gas or solvent at a temperature is from about 0° to about 25° C. (from about 32 to about 75° F.) or any temperature therebetween, or from about 2° to about 15° C. (from about 35 to about 55° F.), or any temperature therebetween, and at a pressure from about 500 to about 1,500 psi or any pressure therebetween, or about 800 to about 1,000 psi any pressure therebetween. Non-limiting examples of supercritical fluid extraction involves loading rates of about 1-10 volumes of liquefied gas at a temperature of above 25° C. (80° F.) and a pressure above 1,100 psi, for example from about 2,000 to about 10,000 psi, or from about 5,000 to about 8,000 psi or any pressure therebetween, or about 800 to about 1500 psi any pressures therebetween.

The medicagenic acid saponin may be present in any suitable amount to provide one or more of the effects described herein. For example, the medicagenic acid saponin may be present in the foodstuff in an amount of about 0.02 to about 10 gram per portion of foodstuff, or any amount therebetween, in an amount of about 0.35 to about 5 gram per portion of foodstuff, or any amount therebetween, in an amount of about 0.05 to about 1 gram per portion of foodstuff, or any amount therebetween, or in an amount of 0.02, 0.04, 0.06, 0.08, 0.1, 0.15, 0.20, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 gram per portion of foodstuff, or any amount therebetween. The medicagenic acid saponin may be present in the foodstuff in an amount of about 5% to about 80% by weight, or any amount therebetween, based on the foodstuff, in an amount of about 10% to about 60% by weight, or any amount therebetween, based on the foodstuff, in an amount of about 10% to about 40% by weight, or any amount therebetween, based on the foodstuff, or in an amount of 5, 10, 15, 20, 25, 30, 35, 40, 45, 50, 55, 60, 65, 70, 75, 80% by weight, or any amount therebetween, based on the foodstuff.

A wide range of medicagenic acid saponins may be useful in and may be incorporated in the foodstuff. In one aspect about 30% to about 100%, or any amount therebetween, of the medicagenic acid saponin present in the foodstuff is selected from the group consisting of 3-GlcA 28-Xyl-Rha-Ara-MA (Medicagenic Acid), 3-GlcA 28-Rha-Ara-MA, and a combination thereof. The medicagenic acid saponin present in the foodstuff may be exclusively 3-GlcA 28-Xyl-Rha-Ara-MA (Medicagenic Acid), 3-GlcA 28-Rha-Ara-MA, and combinations thereof, thus in one aspect the medicagenic acid saponin present in the foodstuff is selected from the group consisting of 3-GlcA 28-Xyl-Rha-Ara-MA (Medicagenic Acid), 3-GlcA 28-Rha-Ara-MA, and a combination thereof.

In a further aspect the present invention provides a preparation comprising from about 25% to about 100% by weight medicagenic acid saponin, or any amount therebewteen.

In a further aspect the present invention provides a preparation comprising from about 80% to about 100% by weight medicagenic acid saponin, or any amount therebewteen.

In a further aspect the present invention provides a method of producing a preparation comprising medicagenic acid saponin from plant material comprising:
(i) providing a solution of said plant material;
(ii) filtering said solution to obtain a filtrate;
(iii) performing solid phase extraction (SPE) on said filtrate to obtain a SPE elute;
(iv) performing chromatography on said SPE elute to produce said preparation.

In a further aspect the present invention provides a method of managing hypercholesterolemia or hyperlipidemia in an animal comprising administering a preparation comprising a medicagenic acid saponin to the animal.

In a further aspect the present invention provides a medicagenic acid saponin for use in medicine.

In a further aspect the present invention provides use of a medicagenic acid saponin in the manufacture for the treatment of hypercholesterolemia or hyperlipidemia.

In each of the above further aspects, preferably the medicagenic acid saponin is bidesmosidic. More preferably the medicagenic acid saponin has sugar moieties on carbon 3 and 28.

The present invention will be further illustrated in the following examples.

### Examples

### Example 1

### Preparation and Analysis

### Plant Material

Alfalfa was grown in France during the summer. The biomass was harvested and dried in the field by pulse air at 40° C. The dry biomass was ground to pieces of about 1 mm using a grinder to produce an alfalfa flour.

### Medicagenic Acid Saponin Extraction And Purification

Alfalfa flour (3.5 kg) was suspended in 52.5 L of a solution composed of 80% of ethanol (95%) and 20% water with a solid: liquid ratio of 1:15. The suspension was stirred for 2 hours at reflux and press-filtered prior to collecting the extract. A second filtration through a 20 μm filter was performed to remove any residue; then ethanol was evaporated at atmospheric pressure. The distillation was interrupted when the vapours reached a temperature of 94° C. The filtrate was then filtrated a second time using 20 μm filter to obtain the saponins concentrated extract. The filtrate (30° C.) was loaded on a solid phase extraction (SPE) column (1000 g silica C18-10% carbon; Silicycle, Quebec, Canada), which had been preconditioned using 6 L of 95% ethanol, followed by 6 L of 15% ethanolic (15% of 95% ethanol) solution, by volume. The column was washed with 12 L of 30% ethanol followed by elution of saponins using 24 L of 50% aqueous ethanolic solution. The elute was evaporated to dryness under vacuum to produce an SPE eluate.

For separation of medicagenic acid saponins from other saponins and plant components a semi-preparative high performance liquid chromatography system was used. Waters' multisolvent delivery system model 60F with a model 600 controller and sample manager model 2767 (Waters Corporation, Milford, Mass.), equipped with a UV photodiode array detector model 2996, were used. The instruments were controlled using MassLynx® software (version 4.0; Waters Corporation, Milford, Mass.). A 200 mg quantity of the dried SPE eluate was diluted in 1 mL of an 80% methanolic aqueous solution for HPLC injection on a Spherisorb S 10 ODS 1, 250×20 mm, 10 μm (Waters Corporation, Milford, Mass.) column. The column was equilibrated with 80% water (eluant A) and 20% acetonitrile (eluant B), both containing 0.01% acetic acid. The elution conditions applied were: 0-2 min isocratic in initial conditions; 2-30 min linear gradient, 20-39% eluant B; 30-50 min linear gradient 39-45% eluant B; 50-56 min linear gradient 45-98% eluant B. The eluants were delivered at a flow rate of 14 mL/min and the system operated at room temperature. The effluent was monitored at 206 nm, and 7-mL fractions were collected, on a time base, in a Waters' fraction collector. Under these conditions, medicagenic acid saponins of interest eluted from about 29.5 to 32.5 min. As would be evident to one of skill in the art, different elution conditions will produce different elution times.

The ethanolic fraction yielded a concentrated saponin extract containing 7-15% (w/w) in total saponins. This extract was further purified using SPE to yield 30 g of solid containing 50% saponins and 50% phyto-estrogens (data not shown). Medicagenic acid saponins were isolated from this preparation by HPLC to yield 7 g of solid containing greater than 80% medicagenic acid saponins.

### Medicagenic Acid Saponin Analysis

Analytical LC-MS analysis was conducted on fractions collected from semi-preparative HPLC chromatography. Waters' Bioseparations module 2796 (Alliance Bio) or 2695 (Alliance) liquid chromatography systems (Waters Corporation, Milford, Mass.), equipped with a quaternary solvent delivery system, vacuum degasser unit, UV photodiode array detector model 2996 and temperature controlled autosampler, were used. Analytical chromatographic separation was performed on a Bakerbond RP-C18 column (4.6×250 mm i.d., 5 μm; J. T. Baker, Phillipsburg N.J.). The mobile phases consisted of 80% water (eluant A) and 20% acetonitrile (eluant B), both containing 0.01% acetic acid. The elution conditions applied were: 0-2 min isocratic in initial conditions; 2-40 min linear gradient, 20-55% eluant B; 40-56 min linear gradient 55-98% eluant B. The eluants were delivered at a flow rate of 0.8 mL/min and the system operated at 25° C.

For saponins confirmation, an Alliance Bio, coupled to a quadrupole time-of-flight (Q-TOF micro) mass spectrometer (Waters Micromass, Manchester, UK) equipped with a Z-spray ion source, were used. Spectra readings were collected in negative ion mode with electrospray ionisation (ESI) interface for mass analysis and detection. All data were acquired in continuum mode over the m/z range of 100-1998. Instrument resolution was better than 5000 FWHM and typical instrument source conditions were as follows: capillary voltage 3 kV, cone voltage 30 V, extraction cone voltage 2 V, source temperature 130° C., desolvation temperature 450° C. and desolvation and nebuliser gas flows of 450 L/hour and 69 L/hour, respectively. The reference probe of the LockSpray® source was set up to infuse a solution containing raffinose [M−H]^{1x}=503.1612 amu which was delivered every 60 seconds at a mean flow rate of 10 mL/min via an external pump (Harvard 11plus, Holliston, Mass.). The HPLC effluent stream was split ˜1:2.7 with the smaller fraction of the stream diverted into the mass spectrometer. Post-column addition of an ammonia solution at 0.1% was allowed to flow into the mass spectrometer at a rate of 3 μL/min via a T junction. The scanning rate was 1 scan/second. The mass spectrometer was controlled using MassLynx® software (version 4.0 SP4, SNC 519; Waters Corporation, Milford, Mass.) and data processed with QuanLynx® option.

### Results

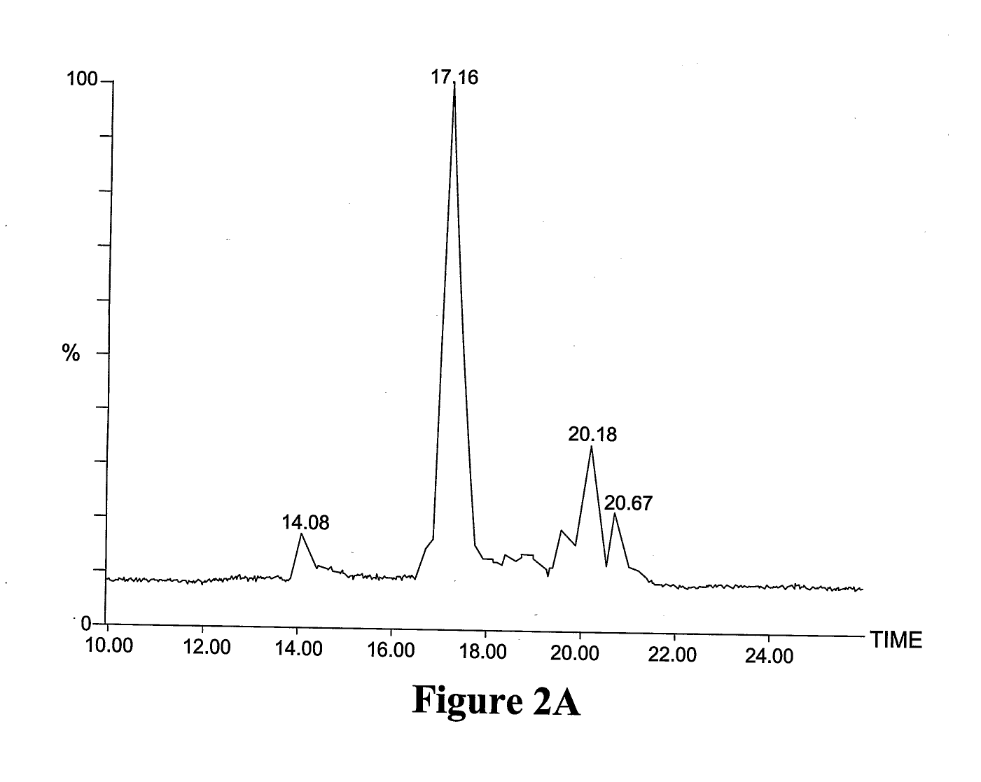

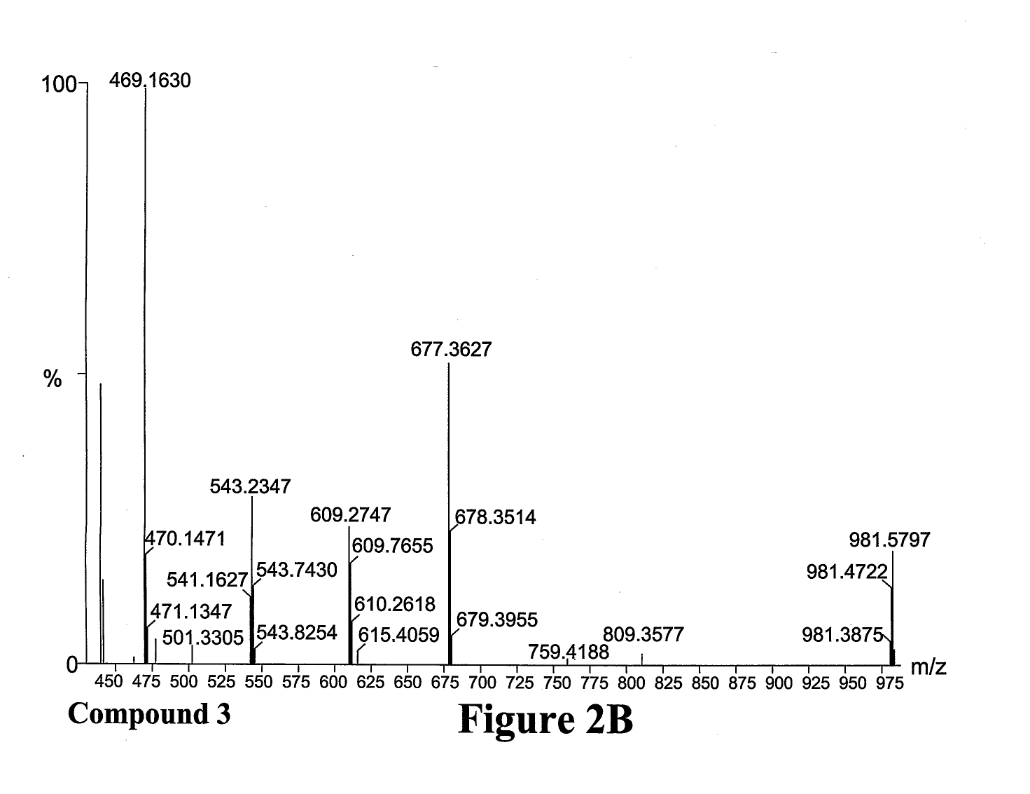

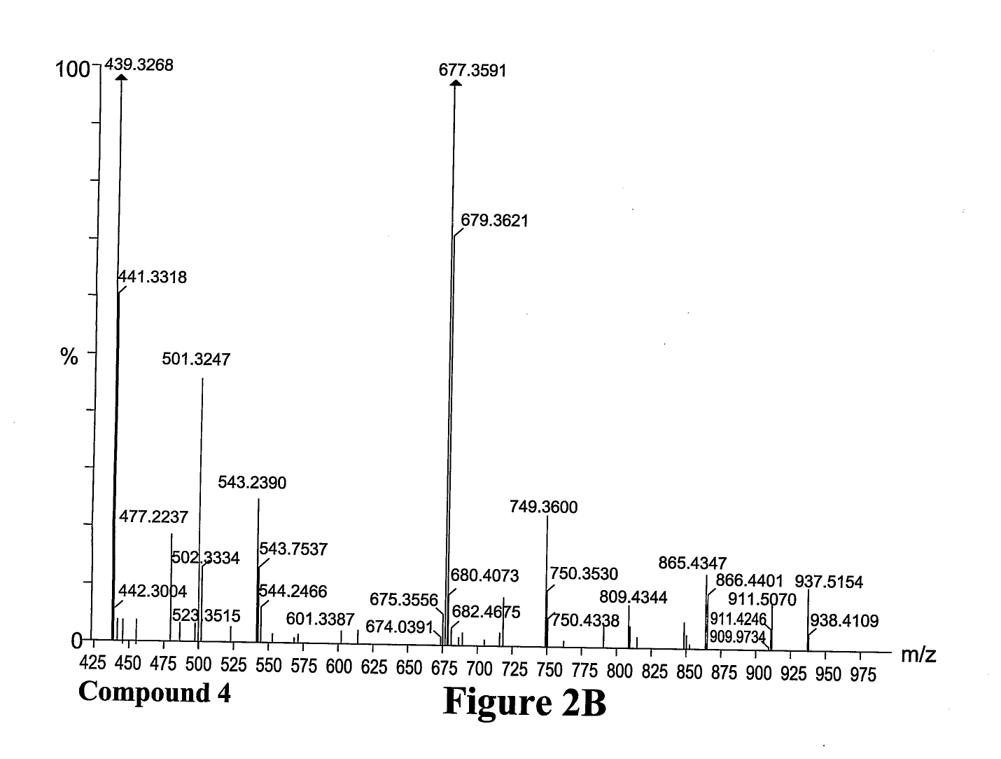

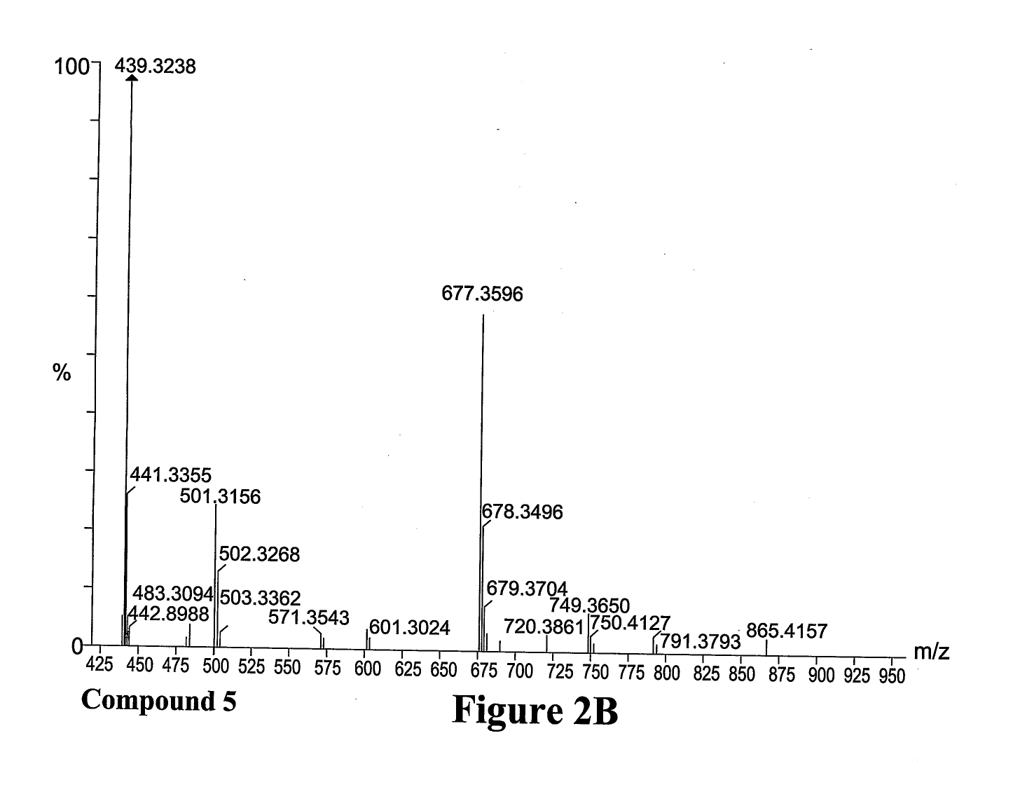

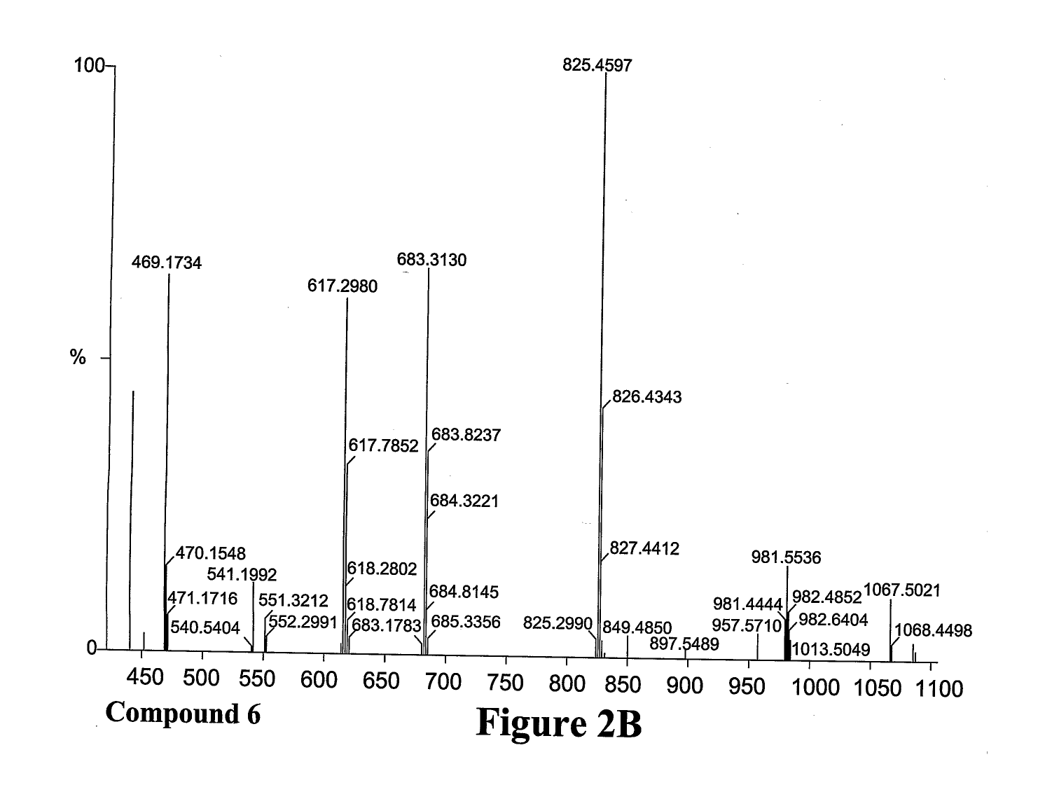

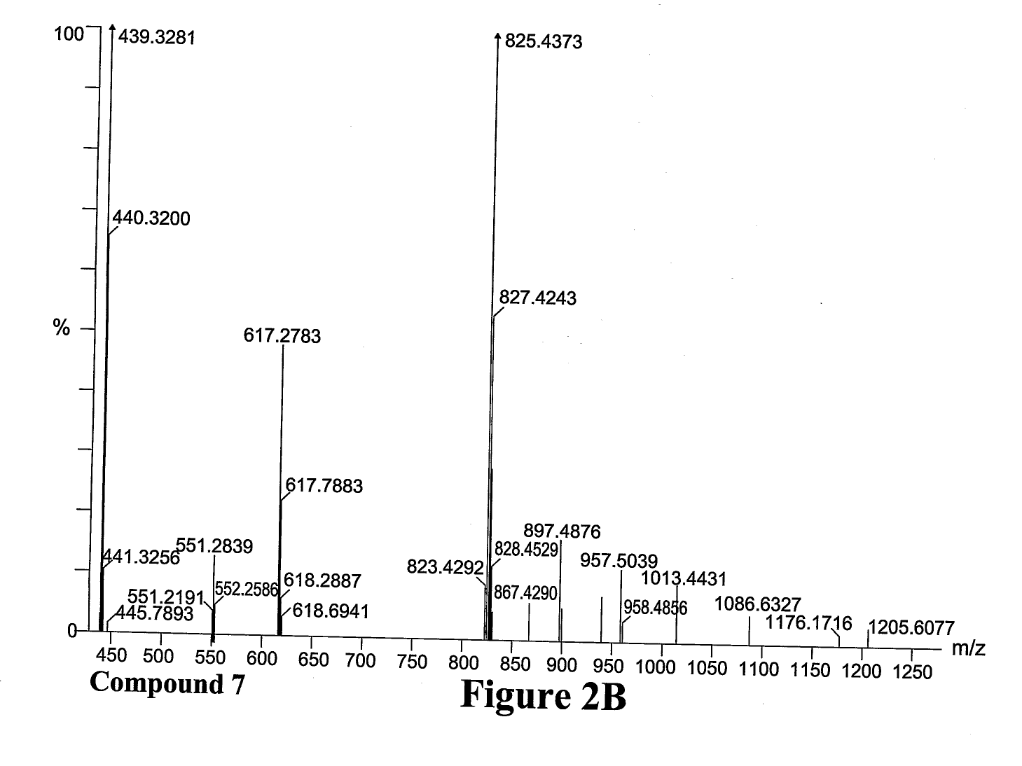

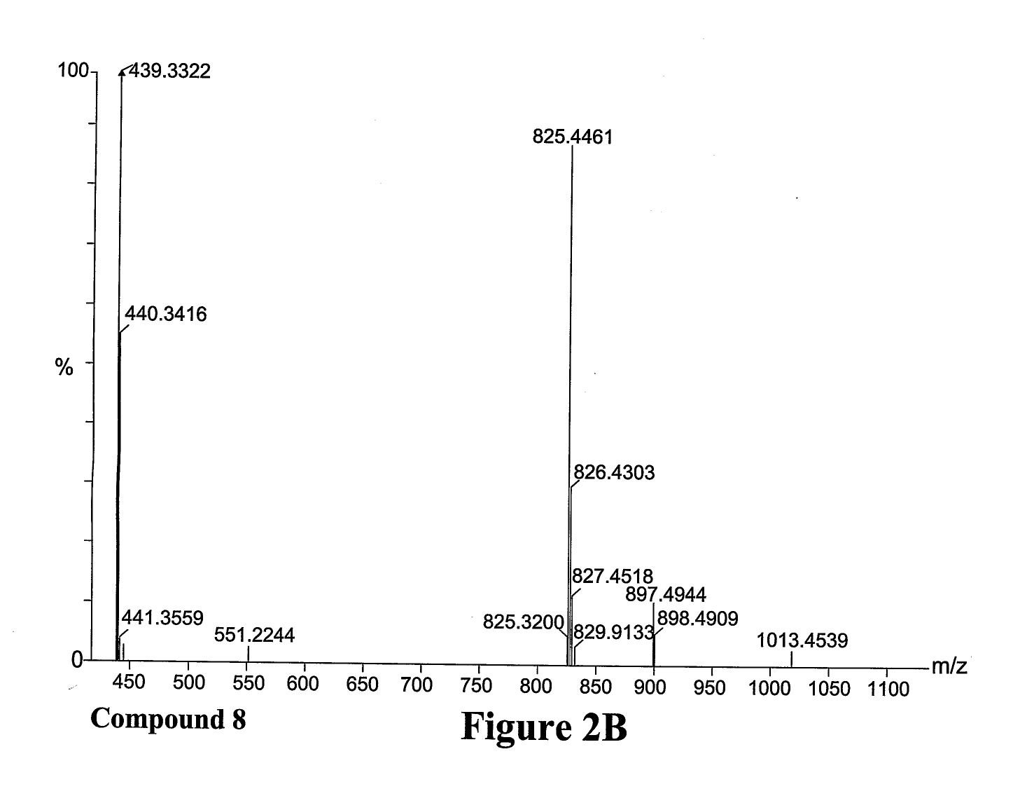

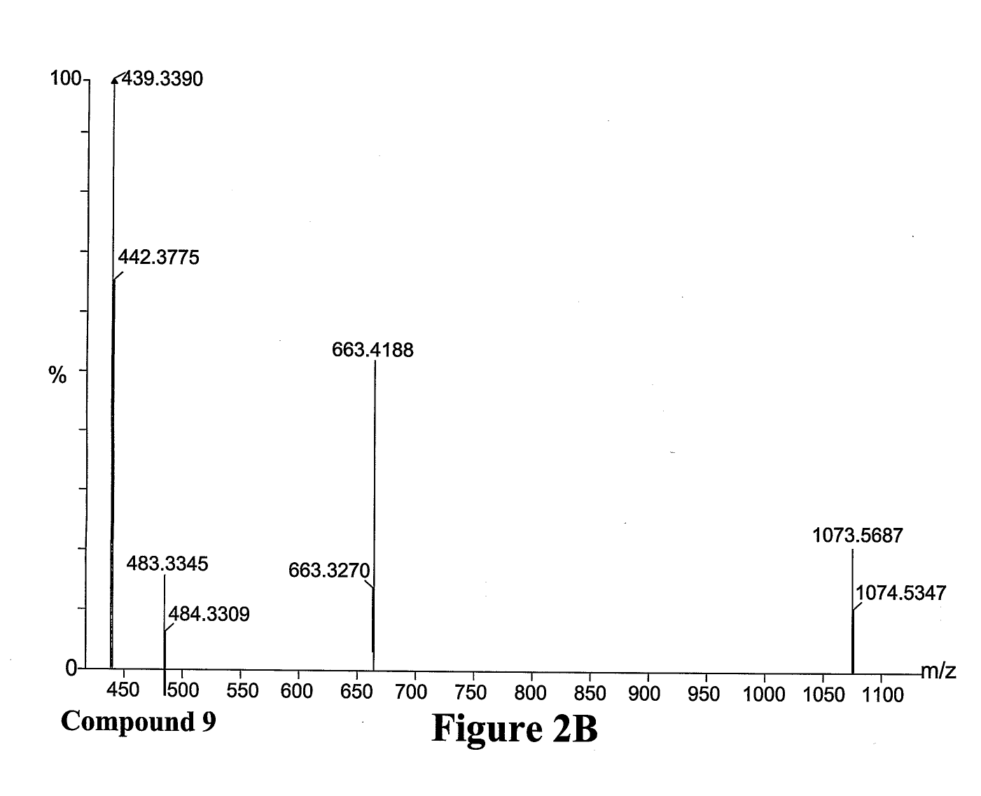

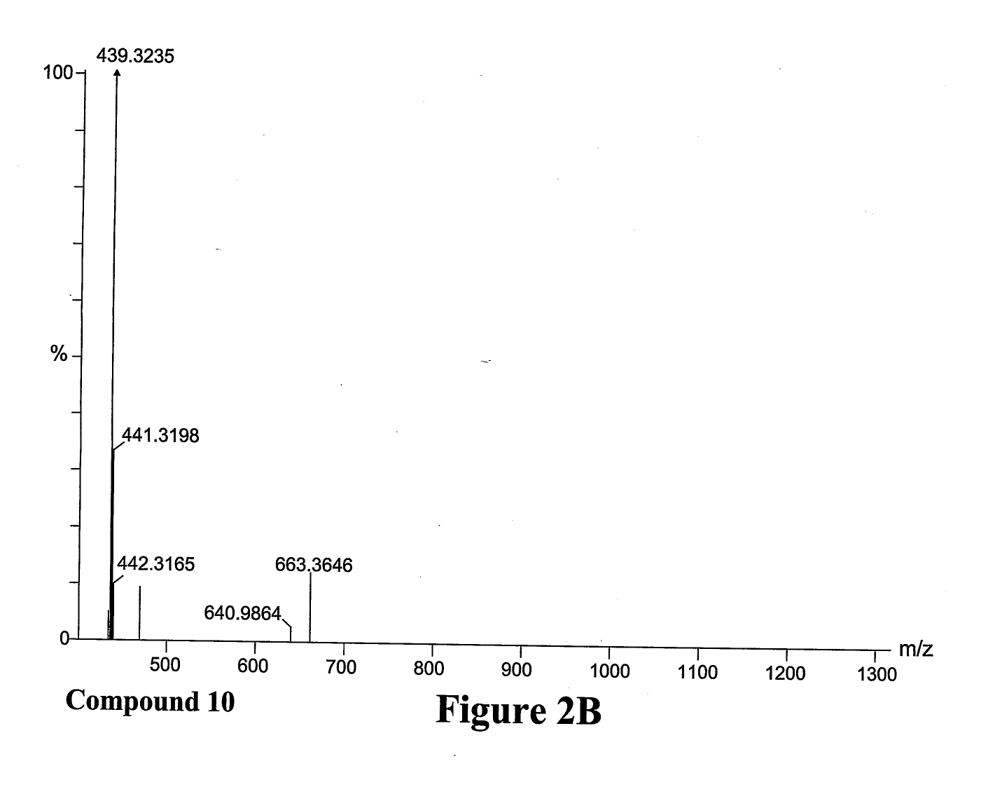

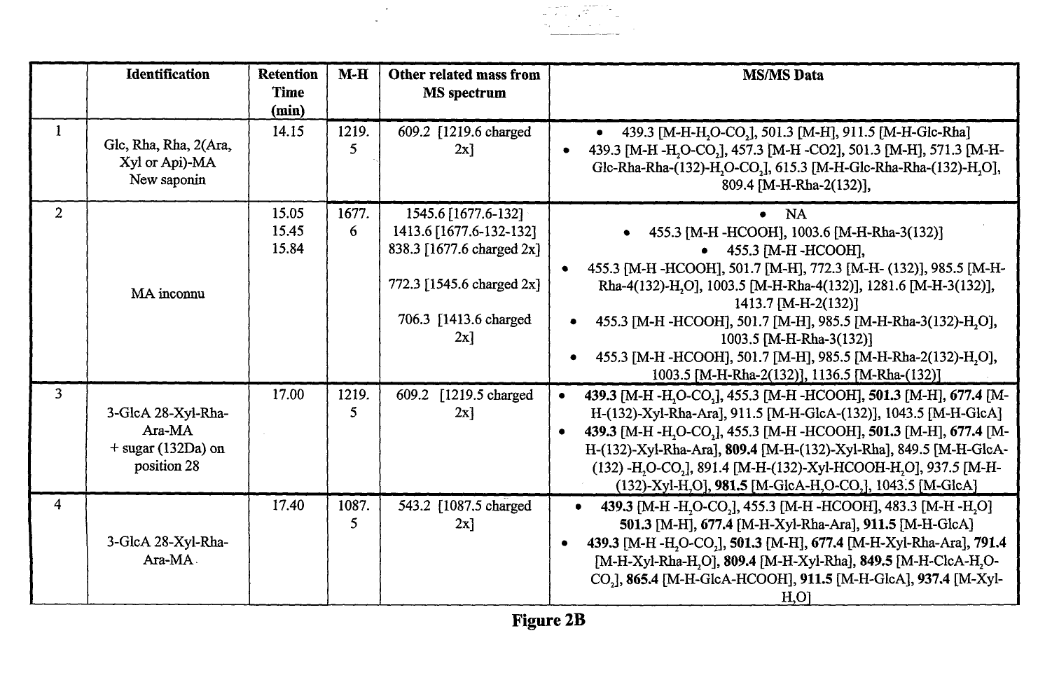

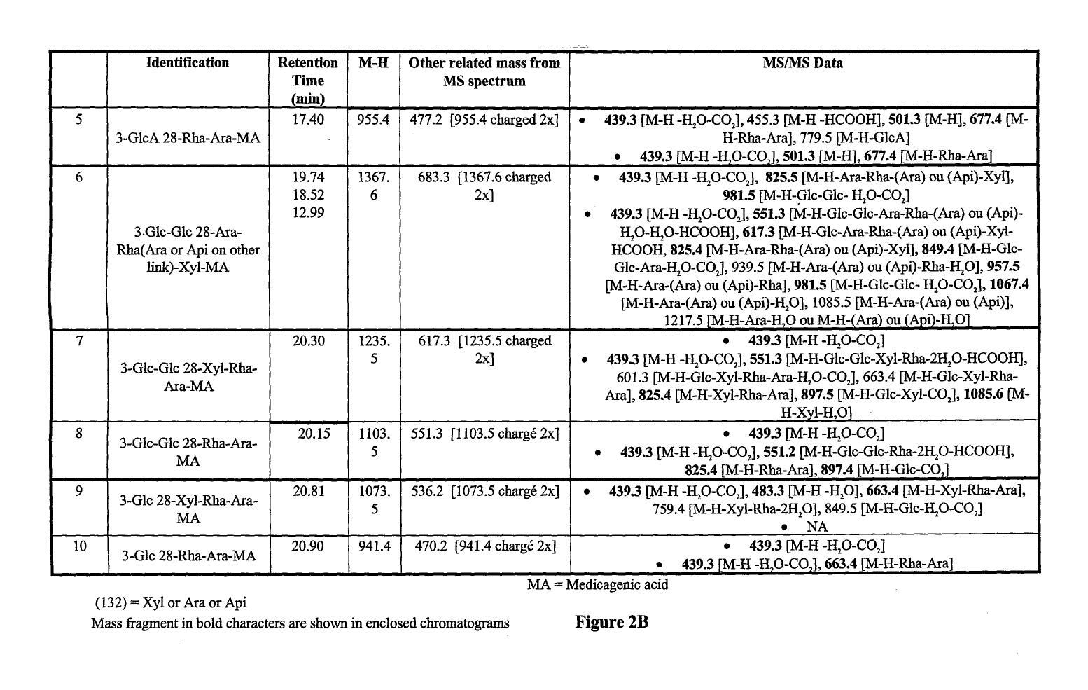

The final HPLC product contained greater than 80% of medicagenic acid saponins, and did not contain any phyto-estrogen coumestrol (data not shown). FIG. 2 shows the chromatographic analysis by mass spectrometry.

Table 2 shows the structure of the different medicagenic acid saponins that were purified and used to conduct the animal studies described below. Figure Y is a detailed description of the fragmentation pattern obtained by mass spectrometry. The majority, 68%, of the identified saponins were attributed to 3-GlcA 28-Xyl-Rha-Ara-MA (Saponin 1 in Table 2; MA: medicagenic acid) and 3-GlcA 28-Rha-Ara-MA (Saponin 2 in Table 2).

TABLE 2
Isolated medicagenic acid saponins (see FIG. 1 for compound structure)

| | MW | | | | |
|---|---|---|---|---|---|
| Saponin | (Da) | R₁ | R₂ | R₃ | R₄ |
| 1 | 1088 | H | GlcA | H | Xyl-Rha-Ara |
| 2 | 955 | H | GlcA | H | Rha-Ara |
| 3 | 1367 | H | Glc-Glc | H | 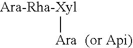 |
| 4 | 1235 | H | Glc-Glc | H | Ara-Rha-Xyl |
| 5 | 1103 | H | Glc-Glc | H | Ara-Rha |
| 6 | 941 | H | Glc | H | Ara-Rha |
| 7 | 1073 | H | Glc | H | Ara-Rha-Xyl |

| | | |
|---|---|---|
| 8 | 1219 | Positions uncharacterized: |
| | | Glc, Rha, Rha, 2(Ara, Xyl or Api) or; |
| | | GlcA, Xyl-Rha-Ara, 1(Ara, Xyl or Api) |
| 9 | 1172 | Uncharacterized medicagenic acid saponins |

### Example 2

### Effect of medicagenic Acid Saponins on Diet Induced Hypercholesterolemia and Moderate Hypercholesterolemia

### Diet

A High Fat High Cholesterol (HFHC) diet used a HFHC feed purchased from Research Diets (New Brunswick N.J.). The diet used to induce hypercholesterolemia in animals contained 0.5% cholesterol and 20% of energy as fat (3% soybean oil and 17% cocoa butter).

The Moderate Cholesterol diet contained 0.25% cholesterol and 12% of energy fat.

Control animals were fed with a regular rodent diet containing no cholesterol.

### Animals

Male golden Syrian hamsters, 29-32 days old and weighing about 85-110 g, were obtained from Charles River (USA). In the first experiment the animals were paired and housed in cages in a temperature-controlled room (20° C.±2° C.) with a 12:12 hour light:dark cycle. In the second experiment, the animals were housed in individual cages. Hamsters were randomly assigned to the following three different groups:
1. Control Group—received normal rodent diet;
2. Saponin Group—were fed with the High Fat High Cholesterol (HFHC) diet or the Moderate Cholesterol diet and received the purified medicagenic acid saponins;
3. Placebo Group—were fed with the High Fat High Cholesterol (HFHC) diet or the Moderate Cholesterol diet and received a placebo of saline.

Hamsters had free access to food and water during the study period. Body weights and food intakes were measured weekly. Food was withdrawn 16-18 hours before blood was collected.

### Experimental Designs

Two different experimental designs were utilized as follows:

### Experiment 1. Diet Induced Hypercholesterolemia

The Saponin Group and Placebo Group were fed with the HFHC diet throughout the experiment and the Control Group received the normal rodent diet. The animals were fed with their respective diets 4 weeks before beginning of treatment with medicagenic acid saponins or placebo, to induce hypercholesterolemia. The Saponin Group of animals then received the medicagenic acid saponins at a dose of 25 mg/kg by gavage once a day for a period of 4 weeks. The Placebo Group of animals received the placebo for the same time period.

### Experiment 2. Diet-induced Moderate Hypercholesterolemia

The Saponin Group of animals received medicagenic acid saponins at a dose of approximately 75 mg/kg administered orally mixed with the Moderate Cholesterol diet, whereas the Placebo Group received a placebo instead of saponin. The Control Group received the normal rodent diet. The study duration was 3 weeks. There was no induction period of moderate hypercholesterolemia

### Plasma Lipid Analysis

All plasma lipid parameters were measured using commercial kits from Roche Diagnostics using a technique described by Lemieux et al. [20] with slight modifications.

### Results

Results are expressed as means±SEM. All data was submitted to Student's t-test or to analysis of variance (ANOVA)

At the end of the induction period in the first study, the mean body weight of the hamsters was determined. The mean body weight of the HFHC fed hamsters was 127.4 g ±4.5 g. Two animals were withdrawn from the study because of their abnormal body weight. One animal showed a body weight of 97.9 g, and another a body weight of 162.5 g. These body weights are more than three times the SEM above or under the body weight mean and the animals were judged physiologically different from the other animals fed with HFHC.

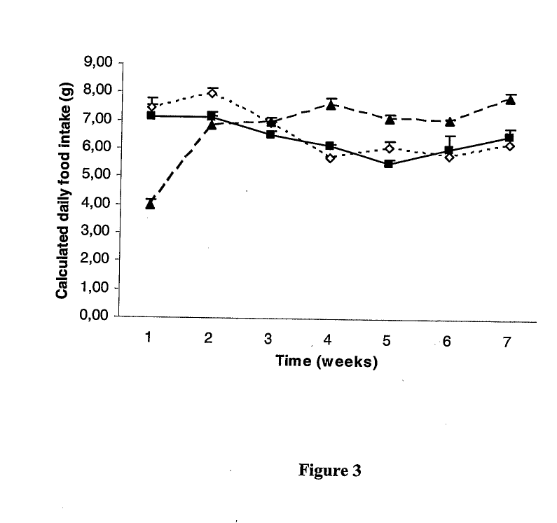

At the end of the experiment there was no difference in gain weight between the Saponin Group, Placebo Group, or the Control Group of animals. The weekly food intake of all groups is shown in FIG. 3.

### Diet Induced Hypercholesterolemia

Total cholesterol, plasma triglycerides and high-density lipoprotein (HDL) were significantly reduced, by 17%, 33% and 11.4%, respectively, in the Saponin Group of hamsters compared to the Placebo Group at the end of the treatment period. There was no statistical difference in non-HDL concentration. Results are shown in FIG. 4.

### Moderate Hypercholesterolemia

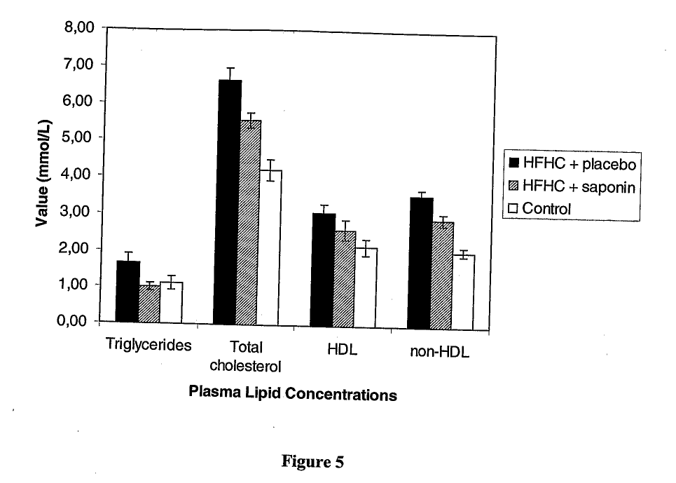

Total cholesterol and non-HDL were significantly reduced, by 16.5%, and 17.5% respectively, in the Saponin Group hamsters compared to the Placebo Group of hamsters. There was a 39% reduction in plasma triglycerides in the Saponin Group, although this was not statistically significant. Results are shown in FIG. 5.

### Wash-Out Period

At the end of the Diet Induced Hypercholesterolemia animal study (Experiment 1), all animals returned to the regular rodent diet containing no cholesterol (control diet). Plasma cholesterol was measured weekly for a period of three weeks.

The Saponin Group of hamsters had significant lower plasma cholesterol levels than the Placebo Group of hamsters for two weeks after the termination of the treatment period. The results are shown in FIG. 6. These results suggest that medicagenic acid saponins have an effect on the elimination of body-synthesized cholesterol.

### Example 3

### Foodstuff Formulation and Evaluation of Taste

Three different products containing different saponins were prepared. Two materials were comparison products and a third comprised the medicagenic acid saponin of the present invention.

Food formulations were prepared and tasted a panel of tasters

Formulation 1—Alfalfa Extract Containing 2% (w/w) in Saponins and around 1% (w/w) in Medicagenic Acid Saponins (Dried Powder)

The formulation was assessed in vegetable drinks, pastries, soup and dairy products. Doses of 3-4 g of this extract per portion were formulated. This corresponds to 60-80 mg in total saponins and 30-40 mg in medicagenic acid saponins. The taste of every food formulation tested was evaluated to be very bitter and persistent. The powder also affected the color and texture of a variety of the foods. The food formulations were tested by 10 people, none of which were able to consume a full portion.

Formulation 2—Alfalfa Extract Containing 50% (w/w) in Saponins and around 25% (w/w) in Medicagenic Acid Saponins (Dried Powder)

This extract was formulated in different fruit juices (orange and banana, grapefruit, cranberry and vegetable drinks) at doses of 100 mg of powder per 250 ml. This dose corresponds to 50 mg of total saponins per portion or 25 mg of medicagenic acid saponins. The drinks were evaluated by 10 people. None of the testers were able to identify the juices containing the formulation. Thus it could this powder can be formulated in food preparations without obvious taste effects. However, this powder contains a significant proportion of coumestrol, a potent phytoestrogen.

Formulation 3—Medicagenic Acid Saponins (>80% Pure) (Dried Powder)

As discussed in the previous example, medicagenic acid saponins led to a decrease in plasma cholesterol and triglycerides.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

1. Kim S-W, Park S-K, Kang S-I, Kang H-C, Oh H-J, Bae C-Y, Bae D-H, Hypocholesterolemic property of Yucca schidigera and Quillaja saponaria extracts in human body, Arch Pharm Res, 26(12), 1042-1046 (2003)
2. Lee S-O, Simons A. L., Murphy P. A., Hendrich S., Soyaysaponins lowered plasma cholesterol and increased fecal bile acids in female Golden Syrian hamsters, Experimental Biology and Medicine, 2005, 472-478
3. Zhao H L, Sim J-S, Shim S H, Ha Y W, Kang S S, Kim Y S, Antiobese and hypolipidemic effects of platycodin saponins in diet-induced obese rats: evidences for lipase inhibition and calorie intake restriction, International Journal of obesity, 2005, 1-8
4. Petit R. P., Sauvaire Y. D., Hillaire-Buys M., Leconte, O. M., Baissac Y. G., Ponsin G. R., Ribes G. R., Steroid saponins from denugreek seeds: extraction, purification, and pharmacological investigation on feeding behaviour on plasma cholesterol, Steroids, vol. 60, 1995, 674-680
5. Matsuura H., Recent advances on the nutritional effects associated with the use of garlic as a supplement, Journal of Nutrition, 2001, 1000S-1005S
6. Kim J. H., Hahm D. H., Yang D. C., Kim J. H., Lee H. J. Shim I., Effect of crude saponin of Korean Red Ginseng on High-Fat Diet Induced Obesity in rats, J. Pharmacol Sci, 97, 124-131 (2005)
7. Massiot G., Lavaud C., Guillaume D., Le Men-Olivier L., Reinvestigation of the sapogenins and prosapogenins from alfalfa, J. Agric. Food Chem., 1988, 36, 902-909
8. Hanson-C H et al, Saponin content of alfalfa as related to location, cutting, variety and other, Bibliography of agriculture, 1963
9. Nowacka J., Oleszek W., Determination of alfalfa (Medicago sativa) saponins by High-Performance Liquid Chromatography, 1994, 42, 727-730
10. Colodny L. R., Montgomery A., Houston M., The role of esterin processed alfalfa saponins in reducing cholesterol, JANA, Vol 3, No. 4, 6-15,2001
11. Beaumont J L, Malinow M R, Houghton D C, et al. Diet-induced systemeic lupus erythematosus (SLE) in primates. Amer J Kid Dis. 1970;1 :345
12. Rashaf G, Gestetner B, Birk Y, et al. Effect of alfalfa saponins on the growth and some aspects of lipids metabolism of mice and quails. J Sci Fd Agric. 1977;30 :2061
13. Molgaard J, von Schenck H, Olsson A. Alfalfa seeds lower low density lipoprotein cholesterol and apolipoprotein B concentrations in patients with type 2 hyperlipoproteinemia. Artherosclerosis. May 1987;65(1-2) :173-179.
14. Ilona Csak. The quantitative effect of Esterin on patients with elevated blood lipid parameters. No. 1 Kutvolgyi Hospital, Semmelweis Medical University, Budapest, Hungary.1991.
15. Ilona Csak. The quantitative effect of Esterin on patients with elevated blood lipid parameters. Semmelweis Medical University, Budapest, Hungary. 1992.
16. Golub I. Zuglo Diabetes Association, Chronic and Rehabilitation Department. Uzsoki, Budapest, XIV, Hungary 1992.
17. Czeizel A. Clinical control study of the effects of Esterin on blood lipid parameters. WHO Collaborating Center for Control of Hereditary Diseases, Semmelweis Medical University, Budapest, Hungary, 1992.
18. Szigetti E. The quantitative effect of Esterin on patients with elevated blood lipid parameters. Semmelweis Medical University, Budapest, Hungary, 1992.
19. Lee, Sun-Ok. et al. Soyasaponins Lowered Plasma Cholesterol and Increased Fecal Bile Acids in Female Golden Syrian Hamsters, Experimental Biology and Medicine, 2005, 230: 472-478
20. Lemieux C., Gelinas Y., Lalonde J., Labrie F., Cianflone K. Deshaies Y, Hypolipidemic action of the SERM acolbifene is associated with decreased liver MTP and increased SR-BI and LDL receptors, J Lipid Res June 2005;46(6): 1285-94

What is claimed is:

1. A preparation comprising from about 50% to about 100% by weight medicagenic acid saponin.

2. The preparation of claim 1, comprising from about 70% to about 100% by weight medicagenic acid saponin.

3. The preparation of claim 1, purified from Medicago sativa.

4. The preparation of claim 3, wherein from about 30% to about 100% of the medicagenic acid saponin is selected from the group consisting of 3-GlcA 28-Xyl-Rha-Ara-MA (Medicagenic Acid), 3-GlcA 28-Rha-Ara-MA, and a combination thereof.

5. A method of producing a preparation comprising from about 50% to about 100% medicagenic acid saponin from plant material comprising:
(i) providing a solution of said plant material;
(ii) filtering said solution to obtain a filtrate;
(iii) performing solid phase extraction (SPE) on said filtrate to obtain a SPE elute;
(iv) performing chromatography on said SPE elute to produce said preparation.

6. The method of claim 5, wherein the plant material in step (i) is from Medicago sativa.

7. The method of claim 5, wherein the chromatography in step (iv) is performed using a high performance liquid chromatography (HPLC).

8. The method of claim 7, wherein the medicagenic acid saponin preparation is eluted from the HPLC system at about 20 to about 40 minutes using a Spherisorb column, equilibrated with 80% water, 20% acetonitrile, both containing 0.01% acetic acid, and eluted using a gradient at room temperature and at a flow rate of 14 mL/min, the gradient comprising: 0-2 min isocratic (80% water, 20% acetonitrile, both containing 0.01% acetic acid), followed by 2-30 min linear gradient, from 20 to 39% acetonitrile, a 30 to 50 min linear gradient from 39 to 45% acetonitrile, and a 50 to 56 min linear gradient from 45 to 98% acetonitrile.

9. The method of claim 7, wherein the medicagenic acid saponin preparation is eluted from the HPLC system at about 25 to about 35 minutes.

10. A preparation produced by the method of claim 5 comprising from about 50% to about 100% medicagenic acid saponin.

11. A method of managing hypercholesterolemia or hyperlipidemia in an animal comprising administering a preparation comprising from about 50% to about 100% by weight medicagenic acid saponin to the animal.

12. The method of claim 11, wherein the animal is a human.

13. A foodstuff comprising a food material and a medicagenic acid saponin

14. The foodstuff of claim 13 wherein the medicagenic acid saponin is a purified form.

15. The foodstuff of claim 14 where in the medicagenic acid saponin is purified from Medicago sativa.

16. The foodstuff of claim 13 wherein from about 30% to about 100% of the medicagenic acid saponin is selected from the group consisting of 3-GlcA 28-Xyl-Rha-Ara-MA (Medicagenic Acid), 3-GlcA 28-Rha-Ara-MA, and a combination thereof.

17. The foodstuff of claim 13 wherein the medicagenic acid saponin is selected from the group consisting of 3-GlcA 28-Xyl-Rha-Ara-MA (Medicagenic Acid), 3-GlcA 28-Rha-Ara-MA, and a combination thereof.

18. The foodstuff of claim 13 wherein the foodstuff is selected from raw and cooked meat, ready to eat meals, pasta sauces, pasteurised soups, mayonnaise, salad dressings, marinades, oil-in-water emulsions, margarines, low fat spreads, water-in-oil emulsions, dairy products, cheese spreads, processed cheese, dairy desserts, flavoured milks, cream, fermented milk products, cheese, butter, condensed milk products, ice cream mixes, soya products, pasteurised liquid egg, bakery products, confectionery products, fruit products, and foods with fat-based or water-containing fillings, salad dressings, acidic dairy products (including natural cheese, cottage cheese, acidified cheese, cream cheese, yoghurt, sour cream, processed cheese), fruit juices, acidic drinks, alcoholic drinks (including wine and beer), chilled dough and cooked or uncooked bakery products, dairy fillings and toppings for baked goods, surface glazes and coatings for bakery items and other heat-processed items, condiments, dips, purees, pickles, marinades, marinated meat or poultry, breaded meat or poultry, pizza toppings and bases, fast food products, kits for making snacks or meals, kits for making bakery products, pet food, broiler feed and any other acidic, heat-processed and/or fungal fermented food products.

19. A method of producing a foodstuff comprising
(i) providing a food material;
(ii) providing a preparation comprising from about 50% to about 100% by weight medicagenic acid saponin;
(iii) combining the food material and the preparation to provide the foodstuff.

20. The method of claim 19 wherein the foodstuff is selected from raw and cooked meat, ready to eat meals, pasta sauces, pasteurised soups, mayonnaise, salad dressings, marinades, oil-in-water emulsions, margarines, low fat spreads, water-in-oil emulsions, dairy products, cheese spreads, processed cheese, dairy desserts, flavoured milks, cream, fermented milk products, cheese, butter, condensed milk products, ice cream mixes, soya products, pasteurised liquid egg, bakery products, confectionery products, fruit products, and foods with fat-based or water-containing fillings, salad dressings, acidic dairy products (including natural cheese, cottage cheese, acidified cheese, cream cheese, yoghurt, sour cream, processed cheese), fruit juices, acidic drinks, alcoholic drinks (including wine and beer), chilled dough and cooked or uncooked bakery products, dairy fillings and toppings for baked goods, surface glazes and coatings for bakery items and other heat-processed items, condiments, dips, purees, pickles, marinades, marinated meat or poultry, breaded meat or poultry, pizza toppings and bases, fast food products, kits for making snacks or meals, kits for making bakery products, pet food, broiler feed and any other acidic, heat-processed and/or fungal fermented food products.
